(19) United States
(12) Patent Application Publication
(10) Pub. No.: US 2020/0182405 A1
(43) Pub. Date: Jun. 11, 2020

(54) REFUELING STATION FOR SUPPLYING ENERGY CARRIERS TO VEHICLES

(71) Applicant: L2 Consultancy B.V., Bilthoven (NL)

(72) Inventor: Constant Johan Van Lookeren Campagne, Bilthoven (NL)

(21) Appl. No.: 16/637,704

(22) PCT Filed: Aug. 10, 2018
(86) PCT No.: PCT/NL2018/050538

§ 371 Date: Feb. 7, 2020

(30) Foreign Application Priority Data
Aug. 10, 2017 (NL) 2019407
Jul. 17, 2018 (NL) PCT/NL2018/050496

(51) Int. Cl. F17C 5/06; F17C 11/00; H01M 8/04082; C12P 5/02

(52) U.S. Cl. CPC F17C 5/06; H01M 2250/20; H01M 8/04201; C12P 5/023; F17C 2265/065; F17C 2221/012; F17C 2270/0184; F17C 2221/033; F17C 2223/0123; F17C 2225/0123; F17C 2225/036; F17C 2227/0157; F17C 2227/0304; F17C 2265/015; F17C 2265/07; F17C 2270/0139; H01M 2250/10; F17C 11/007

(57) ABSTRACT

Disclosed are a refueling method and station for supplying one or more energy carriers to vehicles, wherein the refueling station is connected to a road network and thereby accessible to road vehicles, wherein the energy carriers comprise hydrogen, the refueling station having in a hydrogen production unit configured to produce hydrogen (H₂) and carbon (C) from a hydrocarbon compound by at least one processing unit configured to produce the hydrogen and carbon by pyrolysis of the hydrocarbon compound, wherein the refueling station is configured to supply a hydrogen powered vehicle with the produced hydrogen.

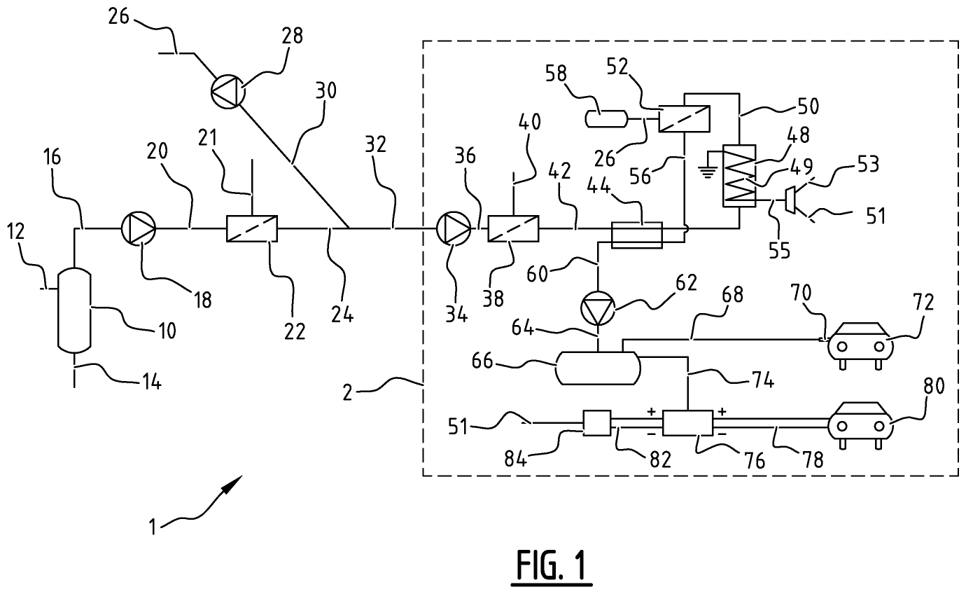

The present patent disclosure relates to a refueling station for supplying one or more energy carriers to vehicles; to a method for supplying one or more energy carriers to vehicles; and to a system comprising a plurality of such refueling stations. Particular embodiments relate to a refueling station for refueling hydrogen-powered vehicles, wherein hydrogen and carbon are produced by pyrolysis of a hydrocarbon compound input.

The use of vehicles such as battery electric vehicles and hydrogen-powered vehicles is increasing. This increased use is partially due to the general belief that these vehicles are zero emission vehicles, that is, that no harmful emissions of greenhouse gases such as carbon dioxide are associated with driving the vehicle. A person using the vehicle generally believes that he causes no harm to the environment by refueling at a refueling station. In use, these vehicles do not directly emit greenhouse gases.

However, the emission of greenhouse gases is actually not reduced, as the electricity and hydrogen are produced centrally. The production of hydrogen and electricity from fossil fuels and transportation to the refueling station will result in greenhouse gas emissions.

The present patent disclosure is related to reducing carbon dioxide emissions of refueling stations that supply energy carriers to cars.

U.S. Pat. No. 7,910,258 B2 discloses a direct carbon fuel cell. Natural gas is used as a feedstock to produce co-products hydrogen gas and electricity by combining molten-salt methane decomposition with the direct carbon fuel cell in one unit. An anode compartment of the fuel cell acts as the methane decomposer to form particulate solid carbon, which remains in the molten salt. Hydrogen gas is evolved from the reaction and exhausted from the cell at a hydrogen ejection port. Natural gas is introduced through a methane feed port. Carbon in the molten salt electrolyte combines with a carbonate ion and produces electricity for a power circuit between an anodic electrode and a cathodic electrode. Carbon dioxide evolves from the anode compartment of the cell as a gas in a concentrated stream.

WO 00/21878 A1 discloses a device for use in a process system for production of hydrogen and carbon based on natural gas, methane or other organic gases as raw material. Gas containing hydrocarbons is guided through a filter into a heat-insulated reaction chamber and heated by means of electric heating coils or excess heat from other high-temperature processes. The reaction chamber contains finely distributed carbon dust that acts as a catalyst for the collection of solid carbon from the gas. The carbon content gets a decreasing gradient upwards in the reaction chamber, and the gas contains mainly hydrogen at the top. The hydrogen-enriched gas is guided to a separation chamber, where parts of the gas are separated through a membrane filter. It is alleged that both the pyrolysis system and fuel cells utilising the hydrogen as fuel can be designed compactly to fit ordinary vehicles.

WO 99/43608 A1 discloses a method of producing high-purity hydrogen by direct cracking of methane at low temperatures using a silica-supported nickel-copper catalyst. Upon deactivation of the catalyst due to carbon deposition, catalyst activity may be fully restored by regenerating the catalyst in air or steam gasification. Because the pyrolysis process has to be discontinued to clean the catalyst, it is not suitable for the production of hydrogen and carbon at a refueling station.

WO 2011/028233 A2 discloses a method for providing a renewable source of a material resource comprising: providing a first source of renewable energy; providing a first stream of materials from a first materials source; providing an electrolyser coupled to the first stream of materials and the first source of renewable energy, wherein the electrolyser produces a material resource from the first stream of materials through electrolysis; and providing the material resource to a first processor for further processing or use. The first processor may be a solar collector comprising a solar concentration mirror and the step of further processing the material resource may comprise disassociating methane to produce carbon and hydrogen.

According to a first aspect of the present patent disclosure, there is provided a refueling station for supplying one or more energy carriers to vehicles, wherein the energy carriers comprise hydrogen, the refueling station having a hydrogen production unit configured to produce hydrogen (H2) and carbon (C) from a hydrocarbon compound by at least one processing unit, wherein the refueling station is configured to supply a hydrogen powered vehicle with the produced hydrogen.

The pyrolysis of the hydrocarbon compound results in the production of carbon, being solid state, which can be used for other purposes. Pyrolysis is a thermochemical decomposition of the hydrocarbon compound at elevated temperatures in the absence of oxygen. The solid-state carbon can be collected and used and stored in useful products such as construction elements for buildings and vehicles. In this way, the amount of produced carbon dioxide is reduced by at least 10 or 20 times compared to, for instance, steam reforming, or the amount of carbon dioxide may even be zero. Also, expensive carbon dioxide capture can be avoided.

The hydrogen production unit is configured to produce substantially only hydrogen and carbon from the hydrocarbon compound, meaning that the carbon from the hydrocarbon compound is converted to carbon, and not to carbon dioxide. In other words, substantially no carbon dioxide is produced. It may be that some byproducts, such as ethane and ethylene, are produced, e.g. less than 5 mole % of the total amount of reaction products.

A further effect is that the hydrogen is produced on-site at, or within a few kilometers of, the refueling station. No supply of hydrogen via trucks or via a dedicated pipeline is required. The natural gas pipeline grid can be used instead to supply the hydrocarbon compound. The on-site production furthermore allows for a lower amount of hydrogen storage means.

In an embodiment, the refueling station comprises an electricity generator to produce a further energy carrier, being electricity, using the produced hydrogen. The hydrocarbon compound can be supplied by a pipeline grid, such as a natural gas pipeline grid. Transport of gaseous hydrocarbons via pipelines has substantially lower energy losses, e.g. lower than about 0.1%, than electricity grids. The latter have a loss of as much as 8%, depending on the distance over which the electricity is transported. The electricity producing embodiment therefore has the further advantage that the hydrogen, being produced on-site from the hydrocarbon compound, is used to produce electricity.

The refueling station, being connected to the road network, is furthermore readily accessible to road vehicles and preferably integrated in existing refueling stations. Another term used for the present refueling station would be a filling station or hydrogen filling station, as will be understood from the above by a person skilled in the art.

The hydrogen production unit preferably comprises at least one reactor comprising a liquid metal, wherein the hydrocarbon compound is gaseous, wherein the at least one reactor is configured to bubble the gaseous hydrocarbon compound through the liquid metal in order to produce the hydrogen and carbon.

The reactor is preferably configured to be heated to temperatures above at least 500° C. and connected to discharge and storage facilities for carbon and hydrogen.

The hydrogen production unit preferably splits the hydrocarbon compound in a fraction that has an increased content of hydrogen and a fraction that has an increased content of carbon at the elevated temperatures above 500° C. The pyrolysis process of the present patent disclosure may be seen as a hydrocarbon cracking process.

The hydrogen production unit may be placed at a distance from where the actual refueling of vehicles takes place, such that hydrogen can be transferred to the area where vehicles are refueled by at least one pipeline connecting the unit and intermediate storage tanks with this area.

An additional advantage is that the hydrogen produced in the refueling station with pyrolysis can have a high purity. Oxidized carbon compounds such as carbon monoxide have a concentration below 0.1 ppm with the pyrolysis process used in the refueling station. Impurities like carbon monoxide in the hydrogen produced can have a negative effect on the efficiency of fuel cells used often in hydrogen-powered vehicles.

The hydrogen production unit is preferably linked with a pipe or tube to a supply system of the hydrocarbon compound, which may be a gas or a liquid. The hydrocarbon compound may comprise one or more alkanes following the formula CnH2n+2, which, when pyrolysed, is split into nC+2nH2, where n is a integer number. Preferably n has a value between 1 and 4. Most preferably n=1, that is, the hydrocarbon compound is most preferably methane. Even more preferably the methane comprises biomethane.

The supply system may comprise a transportable container for the supply of the hydrocarbon compound. This may be an insulated pressurized container for transport of liquefied natural gas comprising methane. In a preferred embodiment the supply system may comprise a natural gas pipeline distribution system. In an even more preferred embodiment the natural gas pipeline system is connected to a production unit of biomethane for feeding the biomethane in the natural gas pipeline distribution system. Biomethane is defined herein as methane produced from renewable biodegradable materials such as fats or carbohydrates from plant material by fermentation. Seaweed is the preferred biodegradable material. The refueling station is preferably provided with at least one hydrogen storage means for storing hydrogen. The hydrogen storage means is preferably connected to a hydrogen fueling unit configured to refuel the hydrogen-powered vehicle with the produced hydrogen. Preferably, the hydrogen fueling unit is provided with at least one hose or tube provided with a plug that fits on the receiving and of the storage tank of the vehicle.

According to a second aspect of the present patent disclosure, there is provided a method for supplying one or more energy carriers to vehicles, the energy carriers comprising hydrogen, the method comprising: providing a hydrocarbon compound; producing, at a refueling station according to the first aspect, hydrogen and carbon by pyrolysis of the hydrocarbon compound; and refueling, at the refueling station, a hydrogen powered vehicle with the produced hydrogen.

In an embodiment, providing the hydrocarbon compound by the supply system comprises producing methane in an anaerobic fermentation reactor configured to ferment a biodegradable material, wherein the hydrogen and carbon are produced at the refueling station using the produced methane. Methane produced in this way may be referred to as biomethane. The vehicle, using the hydrogen produced from the biomethane, will have a net negative equivalent emission of greenhouse gases the more it drives. Thus, in this embodiment, the emission of greenhouse gases is not only reduced, but even brought to below zero. The carbon dioxide is captured from the atmosphere in the biomass and converted to carbon at the refueling station.

According to another aspect of the invention, there is provided a system comprising a plurality of refueling stations according to the invention.

Further aspects, embodiments, features and/or advantages thereof will become apparent from the description above and below, as well as the clauses and/or dependent claims.

### BRIEF DESCRIPTION OF THE FIGURE

The accompanying drawing is used to illustrate presently preferred non-limiting exemplary embodiments of refueling stations and methods of the present patent disclosure. The above and other advantages of the features and objects of the present patent disclosure will become more apparent and the present patent disclosure will be better understood from the following detailed description when read in conjunction with the accompanying drawing, wherein the FIGURE schematically depicts a flow scheme of a system comprising the refueling station and a hydrocarbon compound supply system according to embodiments of the present patent disclosure.

### DESCRIPTION OF EMBODIMENTS

A refueling system 1 for supplying energy carriers to vehicles comprises a refueling station 2. The refueling station is configured to supply one or more energy carriers to vehicles. The energy carriers comprise hydrogen. The refueling station 2 comprises a hydrogen production unit, comprising a processing unit, which in the embodiment shown is embodied as a liquid metal bubble column reactor 48. In other embodiments, any other processing unit configured to produce hydrogen (H2 gas) and solid-state carbon (C), and substantially no carbon dioxide, is suitable. Preferably, the thermal decomposition of the hydrocarbon compound results in low amounts of produced byproducts such as ethane and ethylene, e.g. lower than about 3%. A catalyst such as a metallic catalyst, e.g. a Pd-, Pt- and/or Ni-based catalyst, may be used to increase the conversion efficiency.

In the shown embodiment, the liquid metal bubble column reactor 48 operates at a temperature above the melting temperature of the metal. The metal is preferably tin, or another metal with a melting temperature above 500° C. The reactor 48 is made of a heat resistant material, such as quartz. A supply of hydrocarbon compound as hydrocarbon feed stream 42 is converted to hydrogen and solid-state carbon by pyrolysis in the hydrogen production unit. Substantially no carbon dioxide is produced. The reactor 48 comprises a bubbling unit configured to disperse the input hydrocarbon as bubbles in the liquid metal at a bottom side of the reactor. The input bubbles will then flow upwards through the liquid metal. Preferably, the hydrocarbon feed stream 42 is substantially methane, i.e. a methane feed stream. The produced carbon may have a purity of between 99% and 100%.

With this pyrolysis process, the amount of hydrogen produced per kWh of used electricity (e.g. used for heating the reactor) can be as much as seven times larger than the amount of hydrogen produced per kWh with electrolysis.

The reactor 48 is heated with a heater 49, which in the present embodiment is fed via electrical connection 55. Connection 55 may be connected to a regional electricity grid 51 or to a connection 53 for providing electrical power to the heater. In case of using the regional electricity grid 51 to supply power, when there is a regional surplus of electricity available from renewable sources, such as wind power or photovoltaic cells, the hydrogen produced and stored at the refueling station can act as an energy buffer. Alternative electrical connection 53 is connected to an electricity generator 76 (further connections not shown). Preferably, the liquid metal is heated to a temperature in a range of 800 to 1600° C., preferably 1000-1400° C., most preferably 1050-1350° C. In these temperatures ranges, the yield of hydrogen increases with increasing temperatures. At 1200° C., there may be a yield of hydrogen of about 80%. At the preferred temperatures, there may be a balance between yield of hydrogen and energy expenditure to operate the reactor 48.

An output stream 50 is output by the reactor 48. The refueling station 2 may comprise a carbon powder separator 52. The carbon produced may be in the form of a cake, or may comprise flakes or flake shaped powder in the size range of 15 μm-20 μm agglomerates, wherein the particle size varies from 40 μm to 100 μm. The output stream 50 is input into the carbon powder separator 52 in order to separate the solid-state carbon from the hydrogen. A carbon stream 26 is led to carbon container 58. A separated hydrogen stream 56 is a further output of the separator 52.

The container 58 for storing carbon may be in a form of bags, for instance having a volume of 0.5-10 m3. Alternatively, the carbon container may be a metallic container, which may be provided with a ceramic porous filter, to prevent loss of fine carbon powder. The metallic container may comprise a liquid comprising a suspension of the carbon powder.

In another preferred embodiment the carbon storage container 58 is removable and can be replaced by a similar tank for transport. The carbon storage tank can be provided with filter to contain carbon powder during filling, transport and discharge and separate the hydrogen produced from the carbon powder.

The refueling station 2 may be provided with a heat exchanger 44 to recover and transfer the heat between methane feed stream 42 and the hydrogen stream 56. The latter, coming from the reactor 48 has a substantially higher temperature than the methane feed stream 42. A cooled hydrogen stream 50 exits the heat exchanger 44, and is optionally compressed by compressor 62. The compressed hydrogen stream 64 is then input into at least one hydrogen storage container 66.

The hydrogen may be stored by chemical storage or physical storage systems. The at least hydrogen storage container may use chemical storage, and may be a container comprising ammonia or metal hydrides such as MgH2. In another embodiment the container may be a pressurized container containing hydrogen and a hydrocarbon solvent, that absorbs hydrogen at elevated pressures. Suitable hydrocarbon compounds are methanol, ethanol or butanol, all of which are preferably produced by fermentation of biodegradable materials. The hydrocarbon solvents comprising absorbed hydrogen may be supplied by the refueling station following the invention as a fuel for vehicles with an adapted combustion motor and an adapted fuel tank, making it possible for these vehicles to drive with a low or negative emission of CO2. In another embodiment of a chemical hydrogen storage system the hydrogen container 66 may comprise a carbohydrate. In an embodiment of a physical hydrogen storage system the at least one hydrogen container 66 may be a cryo-compressing storage system.

The hydrogen stored in the hydrogen container may be fed via gas line 68 to a hydrogen powered vehicle 72 using a hose 70 configured to connect to a feed plug of the hydrogen powered vehicle 72. Optionally, the hydrogen may be further compressed, for instance to a pressure in a range of 600 to 900 bars, before fueling the vehicle 72.

In the case, that the hydrocarbon compound is a gas, the refueling station may be provided with a gas purification unit 38. The gas purification unit is configured to purify the gas stream 32 received by the refueling station. The gas stream 32 may be compressed in compressor 34, to form a compressed gas stream 36. The gas purification unit 38 is preferably configured to remove oxygen and oxidized compounds, such as CO and CO2. In case the gas stream 32 supplied to the refueling station 2 comprises nitrogen, the gas purification unit may be provided with a pressure swing adsorption unit comprising a zeolite adsorbent to reduce the nitrogen content.

In a preferred embodiment, the refueling station is provided with an electricity generator 76 powered by the hydrogen produced on-site and connected to the electric power distribution system via connection 51. A hydrogen stream 74 from the hydrogen storage container 66 is input into the electricity generator 76. The generator 76 may be a gas turbine or a fuel cell, both configured for controlled oxidation of hydrogen to generate electricity. In a preferred embodiment, generator 76 is a fuel cell.

The refueling station may be configured to supply direct current (DC) electricity produced by the fuel cell 76 to a battery electric vehicle 80 via electrical connection 78. Furthermore, the DC electricity may be fed via DC connection 82 to a DC/AC converter 84. have a capacity to handle and charge a large number, e.g. larger than 20, preferably larger than 50, of batteries of electric vehicles placed on long term parking places at a distance from the fuel station having a connection to the same electric power distribution system. The effect is that energy loss during transport of electricity over long transmission lines is avoided and emission of carbon dioxide can be reduced. The electricity generator can be a fuel cell or a turbine generator powered by hydrogen gas.

The refueling station 2 is connected to a supply system. The hydrocarbon source to the refueling station 2, i.e. the hydrocarbon gas stream 32, may be supplied via a connection 26 from a natural gas pipeline distribution system. Optionally, the natural gas in connection 26 may be compressed by compressor 28 to form compressed natural gas stream 30 and selectably fed to the refueling station via gas stream 32.

In a more preferred embodiment the hydrocarbon source may be selected to be biomethane, supplied via biomethane pipeline 24. The carbon originating from the biomethane is defined as biocarbon. The effect is, that for every amount of the energy carrier used for transport an amount of carbon dioxide is removed from the atmosphere and can be used and stored in a useful construction element or product produced by for instance three dimensional printing or spray casting using carbon powder as a filler and if necessary carbon fibers for providing mechanical strength.

To this end, biomethane may be produced according to embodiments of the supply system of the present patent disclosure, in a fermentation system. The fermentation system may be provided with biomass as a biodegradable substrate 12, preferably the biomass is seaweed. The biomass is fermented in anaerobic fermentation reactor 2, to form discharge digestate 3 and biogas stream 16. The biogas stream 16 is optionally compressed in compressor 18. Optionally compressed biogas stream 20 is then preferably purified in biogas purifier 22, preferably embodied as a membrane separation unit 22. The biogas purifier 22 is preferably configure to substantially remove CO2, CO, COS, O2 and/or H2S, which may be present in the biogas. Thereafter, the purified biogas 24 is fed to the refueling station via input gas stream 32.

The refueling station 2, following preferred embodiments, makes it possible to provide one or more energy carriers in the form of hydrogen and/or electricity, making it possible for the hydrogen and electricity powered vehicles to drive with a reduced or negative greenhouse gas emission.

As mentioned above, the hydrogen is produced on-site at, or within a few kilometers of, the refueling station. No supply of hydrogen via trucks or via a dedicated pipeline is required. The natural gas pipeline grid can be used instead to supply the hydrocarbon compound. In many countries, such as The Netherlands, existing refueling stations are already connected to the natural gas pipeline grid. Therefore, these existing refueling stations can be readily adapted to include the hydrogen production unit as described above, which uses natural gas as a feedstock.

According to an embodiment, the hydrogen production unit produces no carbon dioxide. No carbon dioxide is produced due to the use of the pyrolysis reaction, which is done in the absence of oxygen. It may be possible that small or minute amounts of carbon dioxide is produced as an impurity.

According to an embodiment, the refueling station comprises a carbon container; and a carbon powder separator configured to separate the solid-state carbon from the hydrogen, wherein the separated carbon is stored in the carbon container. The separated carbon is led from the carbon powder separator to a carbon container. Since the carbon is removed from the reactor in the solid-state and stored, and since no carbon dioxide is produced in the reactor, no carbon dioxide is emitted into the atmosphere by converting natural gas to hydrogen and carbon when using the refueling station according to the present patent disclosure.

According to an embodiment, the processing unit is configured to produce the carbon in the form of a carbon powder, preferably carbon powder comprising agglomerates having a particle size in the range of 15 to 20 μm. Since the produced carbon has a lower density than the liquid metal, it will float on the liquid metal. These particle sizes have the advantage of avoiding blocking of the surface of the liquid metal, which keeps the bubbling through the top surface relatively calm. With these particle sizes, furthermore the carbon can be readily removed from the reactor. In an embodiment the carbon is skimmed off the surface of the liquid metal in the liquid metal bubble column at time intervals.

Preferably, after removal from the reactor, the produced carbon powder is grinded, milled or pulverized. This has the advantage of providing a broader particle size distribution to the carbon powder.

According to an embodiment, the liquid metal in the liquid metal bubble column reactor comprises Ni. Preferably, the liquid metal comprises at least 20% Ni. The liquid metal may further comprise a metal with a lower melting temperature than Ni, such as Sn, in order to reduce the melting temperature of the liquid metal.

In general, a liquid metal bubble column reactor comprises liquid metal. Since such a reactor is not suitable for electrochemical reactions since the liquid metal conducts electrons, the liquid metal is not a molten electrolyte.

Pyrolysis is a thermochemical decomposition of the hydrocarbon compound at elevated temperatures in the absence of oxygen. Cracking is a process whereby (complex) organic molecules are broken into simpler molecules by breaking of carbon-carbon bonds in these molecules.

It should be appreciated by those skilled in the art that the flow scheme described herein represent conceptual views of illustrative embodiments embodying the principles of the invention.

Whilst the principles of the invention have been set out above in connection with specific embodiments, it is to be understood that this description is merely made by way of example and not as a limitation of the scope of protection which is determined by the appended claims.

### Clauses

1. Refueling station for supplying one or more energy carriers to vehicles, wherein the energy carriers comprise hydrogen, the refueling station having in its vicinity a hydrogen production unit configured to substantially produce only hydrogen (H2) and carbon (C) from a hydrocarbon compound by at least one processing unit, wherein the refueling station is configured to supply a hydrogen powered vehicle with the produced hydrogen.

2. Refueling station according to clause 1, wherein the at least one processing unit is configured to produce the hydrogen and carbon by pyrolysis of the hydrocarbon compound.

3. Refueling station according to clause 2, wherein the at least one processing unit comprises a metallic catalyst to catalyze the pyrolysis of the hydrocarbon compound.

4. Refueling station according to clause 1, 2 or 3, wherein the hydrocarbon compound is a gaseous hydrocarbon compound, wherein the at least one processing unit is at least one reactor comprising a liquid metal, wherein the at least one reactor is configured to bubble the gaseous hydrocarbon compound through the liquid metal in order to produce the hydrogen and carbon.

5. Refueling station according to any one of clauses 1 to 4, comprising at least one hydrogen storage means configured to store the produced hydrogen, wherein the refueling station further comprises a hydrogen fueling unit configured to refuel the hydrogen powered vehicle with the produced hydrogen.

6. Refueling station according to any one of clauses 1 to 5, wherein the produced carbon is in the form of a carbon powder, wherein preferably the carbon powder comprises agglomerates having a particle size in the range of 15 to 20 μm.

7. Refueling station according to any one of clauses 1 to 5, wherein the hydrocarbon compound is at least partially biomethane, wherein the biomethane is methane that is produced in an anaerobic fermentation reactor configured to ferment a biodegradable material.

8. Refueling station according to any one of clauses 1 to 7, further comprising at least one gas purification unit configured to purify the hydrocarbon compound to be pyrolysed by the hydrogen production unit.

9. Refueling station according to any one of clauses 1 to 8, wherein the hydrocarbon compound is a gaseous compound, wherein the refueling station is connected to a natural gas transport pipeline system providing the gaseous hydrocarbon compound.

10. Refueling station according to any one of clauses 1 to 9, the energy carriers further comprising electricity, wherein the refueling station comprises an electricity generator configured to generate electricity with hydrogen as an input, wherein the refueling station is configured supply the generated electricity to an electric vehicle and/or to an electrical grid.

11. Refueling station according to clause 10, wherein the electricity generator comprises a fuel cell configured to generate electricity by electrochemical oxidation of the input hydrogen.

12. Method for supplying one or more energy carriers to vehicles, the energy carriers comprising hydrogen, the method comprising:
providing a hydrocarbon compound;
producing, by a refueling station according to any one of clauses 1 to 11, hydrogen and carbon by pyrolysis of the hydrocarbon compound; and
refueling, at the refueling station, a hydrogen powered vehicle with the produced hydrogen.

13. Method according to clause 12, wherein providing the hydrocarbon compound comprises:
producing methane in an anaerobic fermentation reactor configured to ferment a biodegradable material, wherein the hydrogen and carbon are produced at the refueling station using the produced methane.

1-21. (canceled)

22. A refueling station for supplying one or more energy carriers to vehicles, wherein:
the refueling station is connected to a road network and thereby accessible to road vehicles;
the energy carriers comprise hydrogen, the refueling station having a hydrogen production unit configured to produce hydrogen (H₂) and carbon (C) from a hydrocarbon compound by at least one processing unit configured to produce the hydrogen and carbon by pyrolysis of the hydrocarbon compound; and
the refueling station is configured to supply a hydrogen powered vehicle with the produced hydrogen.

23. The refueling station according to claim 22, wherein the at least one processing unit comprises a metallic catalyst to catalyze the pyrolysis of the hydrocarbon compound.

24. The refueling station according to claim 23, wherein the catalyst is a Ni-based metal catalyst.

25. The refueling station according to claim 22, wherein:
the hydrocarbon compound is a gaseous hydrocarbon compound;
the at least one processing unit is at least one reactor comprising a liquid metal; and
the at least one reactor is configured to bubble the gaseous hydrocarbon compound through the liquid metal in order to produce the hydrogen and carbon.

26. The refueling station according to claim 22, comprising:
at least one hydrogen storage means configured to store the produced hydrogen;
wherein the refueling station further comprises a hydrogen fueling unit configured to refuel the hydrogen powered vehicle with the produced hydrogen.

27. The refueling station according to claim 22, wherein the processing unit is configured to produce the carbon in the form of a carbon powder, preferably carbon powder comprising agglomerates having a particle size in the range of 15 to 20 μm.

28. The refueling station according to claim 27, wherein the refueling station comprises:
a carbon container; and
a carbon powder separator configured to separate the solid-state carbon from the hydrogen;
wherein the separated carbon is stored in the carbon container.

29. The refueling station according to claim 22, wherein:
the hydrogen production unit is linked with a pipe or tube to a supply system for supplying the hydrocarbon compound and the hydrocarbon compound is at least partially biomethane; and
the biomethane is methane that is produced in an anaerobic fermentation reactor configured to ferment a biodegradable material.

30. The refueling station according to claim 22, further comprising at least one gas purification unit configured to purify the hydrocarbon compound to be pyrolysed by the hydrogen production unit.

31. The refueling station according to claim 22, wherein:
the hydrocarbon compound is a gaseous compound; and
the refueling station is connected to a natural gas transport pipeline system providing the gaseous hydrocarbon compound.

32. The refueling station according to claim 22, the energy carriers further comprising electricity, wherein:
the refueling station comprises an electricity generator configured to generate electricity with hydrogen as an input; and
the refueling station is configured supply the generated electricity to an electric vehicle and/or to an electrical grid.

33. The refueling station according to claim 32, wherein the electricity generator comprises a fuel cell configured to generate electricity by electrochemical oxidation of the input hydrogen.

34. The refueling station according to claim 22, further comprising:
a hydrogen storage container for storing the hydrogen;
wherein the hydrogen storage container is a pressurised container containing the hydrogen and a hydrocarbon solvent, e.g. methanol, ethanol or butanol, optionally produced by fermentation of biodegradable materials.

35. The refueling station according to claim 22, wherein the hydrogen production unit is configured to produce no carbon dioxide.

36. A method for supplying one or more energy carriers to vehicles, the energy carriers comprising hydrogen, the method comprising:
providing a hydrocarbon compound;
producing, by a refueling station, hydrogen and carbon by pyrolysis of the hydrocarbon compound; and
at the refueling station for supplying one or more energy carriers to vehicles, refueling, a hydrogen powered vehicle with the produced hydrogen;
wherein:
the refueling station is connected to a road network and thereby accessible to road vehicles;
the energy carriers comprise hydrogen, the refueling station having in a hydrogen production unit configured to produce hydrogen (H₂) and carbon (C) from a hydrocarbon compound by at least one processing unit configured to produce the hydrogen and carbon by pyrolysis of the hydrocarbon compound; and
the refueling station is configured to supply a hydrogen powered vehicle with the produced hydrogen.

37. The method according to claim 36, wherein providing the hydrocarbon compound comprises:
producing methane in an anaerobic fermentation reactor configured to ferment a biodegradable material;
wherein the hydrogen and carbon are produced at the refueling station using the produced methane.

38. The method according to claim 37, wherein the produced methane is supplied to the refueling station via a natural gas pipeline distribution system.

39. The method according to claim 36, wherein:
the hydrogen is stored in a pressurised container containing the hydrogen and a hydrocarbon solvent, e.g. methanol, ethanol or butanol, the hydrocarbon optionally produced by fermentation of biodegradable materials;
the method optionally further comprising supplying the hydrocarbon solvent comprising absorbed hydrogen as a fuel to a vehicle with a combustion motor and a fuel tank.

40. A system comprising a plurality of refueling stations for supplying one or more energy carriers to vehicles, refueling, a hydrogen powered vehicle with the produced hydrogen, wherein:
each refueling station is connected to a road network and thereby accessible to road vehicles;
each energy carriers comprise hydrogen, the refueling station having in a hydrogen production unit configured to produce hydrogen (H₂) and carbon (C) from a hydrocarbon compound by at least one processing unit configured to produce the hydrogen and carbon by pyrolysis of the hydrocarbon compound; and
the refueling station is configured to supply a hydrogen powered vehicle with the produced hydrogen.

41. The system according to claim 40, wherein the hydrogen production units of the refueling stations are linked with a respective pipe or tube to a natural gas pipeline distribution system for supplying the hydrocarbon compound.

42. System according to claim 41, further comprising at least one anaerobic fermentation reactor configured to produce biomethane by fermenting a biodegradable material, and arranged to feed the biomethane into the natural gas pipeline distribution system.
